(19) United States
(12) Patent Application Publication
(10) Pub. No.: US 2013/0072474 A1
(43) Pub. Date: Mar. 21, 2013

(54) TREATMENT OF DEMENTIA OF ALZHEIMER'S TYPE WITH MASITINIB

(71) Applicants: Jean-Pierre Kinet, Lexington, MA (US); Alain Moussy, Paris (FR); Colin Field, Ecully (FR)

(72) Inventors: Jean-Pierre Kinet, Lexington, MA (US); Alain Moussy, Paris (FR); Colin Field, Ecully (FR)

(73) Assignee: AB SCIENCE, Paris (FR)

(21) Appl. No.: 13/583,482

(22) PCT Filed: Mar. 9, 2011
(86) PCT No.: PCT/EP2011/053568

§ 371 Date: Sep. 7, 2012

Related U.S. Application Data
(60) Provisional application No. 61/312,079, filed on Mar. 9, 2010.

(51) Int. Cl. A61K 31/55; A61K 45/06; A61K 31/496; A61K 31/497

(52) U.S. Cl. CPC A61K 45/06; A61K 31/496; A61K 31/55
USPC 514/215; 514/253.1

(57) ABSTRACT

The present invention relates to the use of masitinib or a pharmaceutically acceptable salt thereof, for the preparation of a medicament for the treatment of dementia of Alzheimer's type, at appropriate dosage regimen.

The present invention relates to the treatment of dementia of Alzheimer's type, comprising administration of masitinib in an appropriate dosage regimen.

### BACKGROUND OF THE INVENTION

Alzheimer's disease (AD) is the most common cause of dementia. A recent worldwide estimate of Alzheimer's disease prevalence suggests that 26.6 million people were living with the disease in 2006 (Brookmeyer et al., 2007). It is predicted that global prevalence of Alzheimer's will quadruple by 2050 to over 100 million, at which time 1 in 85 people worldwide will be living with the disease. More than 40 percent of those cases will be in late stage Alzheimer's requiring a high level of attention equivalent to nursing home care. AD starts with mild cognitive problems, such as memory loss ultimately progressing to the stage where independent living is not possible.

The principal risk factors for developing AD is age; the likelihood of developing Alzheimer's doubles about every five years after age 65, and after age 85, the risk reaches nearly 50 percent. A family history also increases the risk of developing the disease—which may be due to genetics or environmental factors.

The term dementia describes a syndrome characterized by dysmnesia, intellectual deterioration, personality changes and behavioral abnormalities. These symptoms result in social and occupational decline. Dementia can have multiple etiologies and pathophysiologies, and a range of drugs are currently being developed.

There are currently four drugs approved for the treatment of cognitive symptoms of Alzheimer's disease, classified in two groups.
1. Acetylcholinesterase inhibitors: donepezil, rivastigmine and galantamine. These drugs increase cholinergic transmission by inhibiting acetylcholinesterase at the synaptic cleft. They are indicated for the treatment of patients with mild to moderately severe Alzheimer's dementia.
2. NMDA (N-methyl-D-aspartic acid) receptor antagonist: memantine. This drug modulates the effects of pathologically elevated tonic levels of glutamate that may lead to neuronal dysfunction. It is indicated for the treatment of patients with moderate to severe Alzheimer's disease.

None of these approved drugs represent a cure for the disease, and their efficacy is limited and may decrease with time. In addition, undesirable side effects have been reported, notably vomiting and diarrhea.

Therefore, there is a need for other treatment options to slow down as soon as possible the deterioration of cognitive impairment in patients showing onset or development of dementia associated with Alzheimer's disease.

Masitinib is a small molecule selectively inhibiting specific tyrosine kinases such as c-Kit, PDGFR, Lyn, the Fyn tyrosine kinase, and to a lesser extent the fibroblast growth factor receptor 3 (FGFR3) tyrosine kinase activities, without inhibiting other kinases with known toxicities (those tyrosine kinases or tyrosine kinase receptors attributed to possible tyrosine kinase inhibitor cardiac toxicity (including ABL, KDR and Src), are not inhibited at therapeutic doses of masitinib (Dubreuil et al, 2009). Preclinical data show that masitinib blocks the Fyn tyrosine kinase with an IC₅₀ at 165 nM, a concentration attainable in vivo.

The chemical name for masitinib is 4-(4-methylpiperazin-1-ylmethyl)-N-[4-methyl-3-(4-pyridin-3ylthiazol-2-ylamino)phenyl]benzamide—CAS number 790299-79-5, and the structure is shown below.

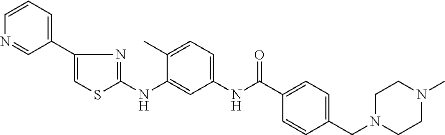 [SMILES: Cc1ccc(NC(=O)c2ccc(CN3CCN(C)CC3)cc2)cc1Nc1nc(-c2cccnc2)cs1]

Masitinib was first described in U.S. Pat. No. 7,423,055 and EP1525200B1. A detailed procedure for the synthesis of masitinib mesilate is given in WO2008/098949.

Aberrations in cell signaling pathways are the cause of many human and animal proliferative diseases and many human inflammatory diseases. Protein tyrosine kinases play a fundamental role in signal transduction and deregulated activity of these enzymes has been observed in cancer, benign proliferative disorders, and in inflammatory diseases. Therefore, specific inhibitors of tyrosine kinases could have potential therapeutic applications in proliferative and inflammatory pathologies.

Protein tyrosine kinases are classified into sub-groups with similar organization and sequence similarity within the kinase domain. They can be associated with receptors in the cell membrane or have an intracellular location. c-Kit is an example of a receptor tyrosine kinase.

Role of c-Kit and Mast Cells in Inflammatory Diseases

Mast cells (MCs) are predominantly found in tissues at the interface between the host and the external environment, such as lung, connective tissue, lymphoid tissue, gut mucosa, and skin. Immature MC progenitors circulate in the bloodstream and differentiate in tissues. These differentiation and proliferation processes are under the influence of cytokines; one of utmost importance is stem cell factor (SCF), also termed Kit Ligand (KL), Steel factor or Mast Cell Growth Factor (MCGF). The SCF receptor is encoded by the proto-oncogene c-Kit.

c-Kit encodes a hematopoietic growth factor (HGF) receptor with tyrosine kinase activity. Kit receptor activity appears essential for the development of melanoblasts, germ cells as well as hematopoietic cells. The W (White Spotting) and Sl (Steel) loci encode Kit and its ligand, SCF, respectively, and mutations in these genes cause pigmentation defects, infertility and deficiencies in the hematopoietic system, including decreased numbers of MCs. It has been shown that SCF regulates the migration, maturation, proliferation, and activation of MCs in vivo. Binding of SCF to the c-Kit receptor induces c-Kit dimerization followed by its transphosphorylation, leading to the recruitment and activation of various intracytoplasmic substrates. These activated substrates induce multiple intracellular signaling pathways responsible for cell proliferation and activation.

“Normal” MC activation is followed by the controlled release of a variety of mediators that are essential for the defense of the organism against invading pathogens. By contrast, if hyperactivation of MCs occurs, uncontrolled hypersecretion of these mediators is deleterious for the body.

In vivo and in vitro studies suggest that human MCs are capable of expressing both pro- and anti-inflammatory cytokines, including TNF-α, IL-3, IL-4, IL-5, IL-6, IL-8, IL-10, IL-12, IL-13, IL-16, GM-CSF, SCF, basic fibroblast growth factor (bFGF), transforming growth factor beta (TGF-β) and many chemokines such as macrophage inflammatory protein 1 alpha (MIP-1α), and monocyte chemotactic protein 1 (MCP-1).

Human MCs constitutively express a number of receptors for different biological molecules. Among these receptors, whose ligation induces the activation of MCs, the best known is the high affinity receptor for IgE (FccRI). Binding of IgE-multivalent antigen complexes to FceRI leads to receptor aggregation and internalization, signaling, and degranulation. This can be accompanied by the transcription of cytokine genes, thus, perpetuating the inflammatory response. Moreover, triggering of MCs leads to the secretion of diverse preformed and/or de novo synthesized mediators, such as vasoactive amines (histamine, serotonin), sulfated proteoglycans, lipid mediators (prostaglandin D2, leucotrienes), growth factors, proteases, cytokines and chemokines as described previously. These mediators can, either alone or in synergy with macrophage- and T cell-derived cytokines, generate a complex inflammatory response and can induce the recruitment and activation of inflammatory cells to the site of degranulation.

MCs thus play a prominent role in all inflammatory processes because they express receptors for molecules that are usually involved in such reactions and because they release large amounts of various mediators that sustain the inflammatory network. Molecules able to inhibit the survival and/or activation of MCs are being tested for the treatment of inflammatory diseases.

Examination of the brains of patients affected with AD reveals two microscopic changes: senile plaques develop between neurons, and neurofibrillary tangles develop within neurons. These changes are thought to be intricately related to the cause, development and course of the disease. Neurofibrillary tangles are made up partly of a protein called tau (τ), which links together to form filaments. The density of these filaments within neurons is directly related to the severity of dementia. It is unclear why tangles form and whether they are linked to plaque formation. Their ultimate effect, however, is compromised microtubular function, with destruction of the neuron.

It is speculated that inflammation around plaques destroys neighboring neurons. Plaques, which are composed of β-amyloid polypeptides are thought to form as a result of disorders in processing β-amyloid and its precursor protein (St George-Hyslop PH, 2000).

Inflammation surrounding β-amyloid plaques with resultant destruction of neurons is thought to be a key factor in the pathogenesis of AD. Observational studies have found that people who regularly use nonsteroidal anti-inflammatory drugs (NSAID) have a lower incidence of AD. Thus, NSAIDs are likely to have a neuroprotective effect. However, several studies of anti-inflammatory drugs do not show a benefit for treatment (Veld B A et al, 2001).

The abnormal phosphorylation of tau protein on serines and threonines is a hallmark characteristic of the neurofibrillary tangles of AD. The discovery that tau could be tyrosine phosphorylated together with evidence that Aβ signal transduction involved tyrosine phosphorylation, suggested that tyrosine phosphorylation of tau occurs during neurodegeneration (Lee et al, 2004). The authors showed that human tau tyr18 was phosphorylated by the Src family tyrosine kinase Fyn. Moreover, immunocytochemical studies indicate that tyrosine phosphorylated tau was present in the neurofibrillary tangles in AD brain. These data add new support for a role for Fyn in the neurodegenerative process (Lee et al, 2004).

In connection with the present invention, we recently discovered that masitinib inhibits the tyrosine kinase Fyn and we searched into its potential for use in the treatment of dementia of AD type.

We then investigated the effect of masitinib in clinical trials at several dosage regimens combined with NMDA (N-methyl-D-aspartic acid) receptor antagonist and/or acetylcholinesterase inhibitors in patients developing dementia associated with AD. We found a protective effect of masitinib allowing slowdown of disease progression in patients, especially in patients having low or moderate dementia of AD type, in particular in patients with MMSE between 12 to 25.

### DESCRIPTION OF THE INVENTION

The present invention relates to the use of masitinib or a pharmaceutically acceptable salt thereof and of at least one of NMDA (N-methyl-D-aspartic acid) receptor antagonists and acetylcholinesterase inhibitors for the preparation of a medicament for the treatment of dementia of Alzheimer's type according to the classification of the Diagnostic and Statistical Manual—Revision 4 (DSM IV criteria) or according to the probable Alzheimer's disease criteria of the National Institute of Neurological and Communicative Disorders and Stroke and the Alzheimer's Disease and Related Disorders Association (NINCDS-ADRDA), in human patients, wherein masitinib is to be administered daily at a starting dose of 3.0 to 6.0±1.5 mg/kg/day, optionally combined with at least one of NMDA (N-methyl-D-aspartic acid) receptor antagonists and acetylcholinesterase inhibitors, and wherein said patients are between 9 to 26 to the mini-mental state examination (MMSE).

The present invention also relates to masitinib or a pharmaceutically acceptable salt thereof and of at least one of NMDA (N-methyl-D-aspartic acid) receptor antagonists and acetylcholinesterase inhibitors as a combined preparation for its use separately, simultaneously or sequentially in time in the treatment of dementia of Alzheimer's type according to the classification of the Diagnostic and Statistical Manual—Revision 4 (DSM IV criteria) or according to the probable Alzheimer's disease criteria of the National Institute of Neurological and Communicative Disorders and Stroke and the Alzheimer's Disease and Related Disorders Association (NINCDS-ADRDA), in human patients, wherein masitinib is to be administered daily at a starting dose of 3.0 to 6.0±1.5 mg/kg/day, optionally combined with at least one of NMDA (N-methyl-D-aspartic acid) receptor antagonists and acetylcholinesterase inhibitors, and wherein said patients are between 9 to 26 to the mini-mental state examination (MMSE).

The invention also relates to a method of treatment of dementia of Alzheimer's type according to the classification of the Diagnostic and Statistical Manual—Revision 4 (DSM IV criteria) or according to the probable Alzheimer's disease criteria of the National Institute of Neurological and Communicative Disorders and Stroke and the Alzheimer's Disease and Related Disorders Association (NINCDS-ADRDA), comprising administering masitinib or a pharmaceutically acceptable salt thereof and at least one of NMDA (N-methyl-D-aspartic acid) receptor antagonists and acetylcholinesterase inhibitors to human patients, wherein masitinib is to be administered daily at a starting dose of 3.0 to 6.0±1.5 mg/kg/day, optionally combined with at least one of NMDA (N-methyl-D-aspartic acid) receptor antagonists and acetylcholinesterase inhibitors, and wherein said patients are between 9 to 26 to the mini-mental state examination (MMSE).

Advantageously, in the therapeutic uses or the combined preparation or the method above, said patients are between 10 to 26 to the mini-mental state examination (MMSE). Also, said patients are preferably between 0.5 and 2 in the CDR scale. Patients according to the invention are those afflicted with dementia of mild to moderately severe Alzheimer's type, more specifically with an MMSE score of 10-26, or 12 to 26, or even 15 to 26.

In one preferred embodiment, masitinib is masitinib mesilate. Regarding best dosage regimen, masitinib is to be administered at a starting daily dose of 3.0 to 6.0 mg/kg/day; nonetheless masitinib can be dose escalated by increments of 1.5 mg/kg/day to reach a maximum of 7.5 mg/kg/day in low responder patients.

Indeed, depending on age, individual condition, mode of administration, and the clinical setting, effective doses of masitinib or a pharmaceutically acceptable salt thereof in human patients are 3.0 to 6.0 mg/kg/day per os, preferably in two daily intakes. For adult human patients with dementia of Alzheimer's type, a starting dose of masitinib or a pharmaceutically acceptable salt thereof of 3.0 to 6.0 mg/kg/day has been found to be the preferred embodiment according to the invention. For patients with an inadequate response after an assessment of response to therapy and in the absence of limiting toxicities, dose escalation of masitinib mesilate or a pharmaceutically acceptable salt thereof to a maximum of 7.5 mg/kg/day can be safely considered and patients may be treated as long as they benefit from treatment and in the absence of limiting toxicities.

If dose escalation is undertaken, it is suggested that the starting dose of 3.0 to 6.0 mg/kg/day be incremented by 1 to 2 mg/kg/day up to a maximum dose of 7.5 mg/kg/day, over a period which depends upon clinical observations. For example, a single dose escalation of masitinib mesilate or a pharmaceutically acceptable salt thereof may take from 1 to 2 months. It is also contemplated herein that to fully obtain the therapeutic benefits of a patient-optimized dose of masitinib or a pharmaceutically acceptable salt thereof, dose increments smaller than 1 to 2 mg/kg/day could be implemented. Dose reduction is to be considered to reduce toxicity in appropriate cases.

Dose adjustment can be considered a dynamic process, with a patient undergoing multiple increases and/or decreases to optimize the balance between response and toxicity throughout treatment, both of which are likely to vary over time and duration of drug exposure.

Any dose indicated herein refers to the amount of active ingredient as such, not to its salt form.

Pharmaceutically acceptable salts are pharmaceutically acceptable acid addition salts, like for example with inorganic acids, such as hydrochloric acid, sulfuric acid or a phosphoric acid, or with suitable organic carboxylic or sulfonic acids, for example aliphatic mono- or di-carboxylic acids, such as trifluoroacetic acid, acetic acid, propionic acid, glycolic acid, succinic acid, maleic acid, fumaric acid, hydroxymaleic acid, malic acid, tartaric acid, citric acid or oxalic acid, or amino acids such as arginine or lysine, aromatic carboxylic acids, such as benzoic acid, 2-phenoxy-benzoic acid, 2-acetoxy-benzoic acid, salicylic acid, 4-aminosalicylic acid, aromatic-aliphatic carboxylic acids, such as mandelic acid or cinnamic acid, heteroaromatic carboxylic acids, such as nicotinic acid or isonicotinic acid, aliphatic sulfonic acids, such as methane-, ethane- or 2-hydroxyethane-sulfonic, in particular methanesulfonic acid (or mesilate), or aromatic sulfonic acids, for example benzene-, p-toluene- or naphthalene-2-sulfonic acid.

In a preferred embodiment of the above-depicted treatment, the active ingredient masitinib is administered in the form of masitinib mesilate; which is the orally bioavailable mesylate salt of masitinib—CAS 1048007-93-7 (MsOH); C28H30N60S.CH3SO3H; MW 594.76:

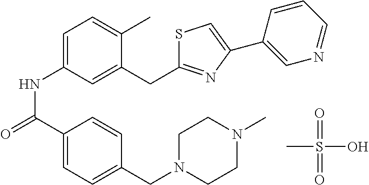

Given that the masitinib dose in mg/kg/day used in the described dose regimens refers to the amount of active ingredient masitinib, compositional variations of a pharmaceutically acceptable salt of masitinib mesilate will not change the said dose regimens.

Masitinib may be administered via different routes of administration but oral administration is preferred. Thus, in still another preferred embodiment, in the therapeutic uses or the combined preparation or the method above, masitinib or salts thereof, is administered orally; preferably twice a day for long term period such as over more than 6 months, preferably more than 12 months. Masitinib can be administered in the form of 100 and 200 mg tablets.

In the present invention as defined above, NMDA receptor antagonist is memantine and the acetylcholinesterase inhibitor is selected from donepezil, rivastigmine and galantamine.

Dosing for memantine (Namenda® or Ebixa®) is in accordance to the manufacture's recommendations (Namenda Label Information, 2007). That is, tablets administered orally at an initial dose of 5 mg once a day; may increase dose to 10 mg/day (5 mg twice a day), 15 mg/day (5 mg and 10 mg as separate doses), and 20 mg/day (10 mg twice a day) at minimum 1 week intervals if well tolerated. Also available as oral solution, administered in same dosage as for tablets. Thus in the therapetutical uses or method as defined above, memantine is to be administered between 5 to 20 mg/day.

Dosing for donepezil (Aricept®) is in accordance to the manufacture's recommendations (Aricept Label Information). That is, tablets administered orally at an initial dose of 5 mg once a day; may increase dose to 10 mg/day after 4-6 weeks if well tolerated.

Dosing for rivastigmine (Exelon®) is in accordance to the manufacture's recommendations (Exelon Label Information, 2006). That is, capsules administered orally at an initial dose of 3 mg/day (1.5 mg twice a day); may increase dose to 6 mg/day (3 mg twice a day), 9 mg (4.5 mg twice a day), and 12 mg/day (6 mg twice a day) at minimum 2-week intervals if well tolerated. Also available as oral solution, administered at the same dosage as for capsules. Also available as a patch administered at an initial dose of 4.6 mg once a day; may increase to 9.5 mg once a day after minimum of 4 weeks if well tolerated.

Dosing for galantamine (Razadyne® or Reminyl®) is in accordance to the manufacture's recommendations (Razadyne Label Information, 2008). That is, tablets administered orally at an initial dose of 8 mg/day (4 mg twice a day); may increase dose to 16 mg/day (8 mg twice a day) and 24 mg/day (12 mg twice a day) at minimum 4-week intervals if well tolerated. Also available as extended-release capsule administered in same dosage as for tablets but taken once a day. Also available as oral solution at the same dosage as above. Thus, in the therapeutic uses or the combined preparation or the method as defined above, donepezil is to be administered between 5 to 10 mg/day, rivastigmine is to be administered between 3 to 12 mg/day, or galantamine is to be administered between 8 to 24 mg/day.

In this regard, masitinib and at least one of NMDA receptor antagonist and acetylcholinesterase inhibitor are to be administered separately, simultaneously or sequentially in time.

The present invention is illustrated by means of the following example.

### EXAMPLE 1

### Clinical Evaluation in Patients

A multicenter, randomized, double-blind, placebo-controlled, parallel-group phase 2 study evaluating the activity of masitinib given orally for 24 weeks has been performed in patients suffering from mild to moderate dementia of Alzheimer's type (protocol period completed, extension ongoing).

Methods

Patients: Patients enrolled in this study had dementia of Alzheimer's type (according to DSM IV criteria), probable Alzheimer's disease according to NINCDS-ADRDA criteria, MMSE≧12 and ≦26, and CDR of 1 or 2. Patients had been receiving a stable dose of acetylcholinesterase inhibitors for a minimum of 6 months and/or memantine for a minimum of 3 months. Exclusion criteria included inadequate organ function (defined via blood test levels), dementia due to any other causes, uncontrolled depression, psychosis, delusions or delirium.

Treatment: In addition to a stable dose of acetylcholinesterase inhibitors and/or memantine, patients received masitinib or placebo, supplied as 100 and 200 mg tablets, per os for 24 weeks. Patients received masitinib or placebo initially at a daily total dose of 3 mg/kg or 6 mg/kg, administered twice daily with meals. Dose could be increased or decreased by 1.5 mg/kg/day depending on efficacy and toxicity assessments. Five patients were allocated to each masitinib dose for every three placebo patients. Treatment with any registered or putative cognitive enhancer or disease modifier (other than donepezil, galantamine, rivastigmine or memantine), or any investigational therapies was not allowed.

Dose Modification or Removal from Therapy:

In the event of insufficient response and manageable toxicity, the dose could be increased by 1.5 mg/kg/day to a maximum of 7.5 mg/kg/day, at 1 and 2 months after treatment.

Depending on the severity and frequency of episodes, treatment was interrupted or discontinued if any of the following occurred: neutropenia, renal or cardiac toxicity, nausea/vomiting, diarrhea, edema, rash.

Patients discontinued the trial in the event of withdrawal of consent, unacceptable toxicity, protocol violation, or a worsening of disease status after 3 months of treatment necessitating a change in treatment. Patients in the treatment extension continued until treatment escape, product registration or development cessation, whichever occurred first

Efficacy and Safety Assessment:

The following efficacy criteria were evaluated after 1, 2, 3 and 6 months of treatment:

ADAS-Cog: Alzheimer's Disease Assessment Scale, a cognitive sub-scale which examines selected aspects of cognitive performance including elements of memory, orientation, attention, reasoning, language and praxis. The ADAS-Cog scoring range is from 0 to 70, with higher scores indicating greater cognitive impairment.

Abbreviations:

CIBIC-plus: Clinician's Interview Based Impression of Change, requires assessment by a skilled clinician based on his/her observation at an interview with the patient, in combination with information supplied by a caregiver familiar with the behavior of the patient over the interval rated. The CIBIC-plus is scored as a seven-point categorical rating, ranging from a score of 1, indicating “markedly improved”, to a score of 4, indicating “no change”, to a score of 7, indicating “marked worsening”

ADCS-ADL: Alzheimer's Disease Cooperative Study-Activities of Daily Living, which measures a wide spectrum of dementia severity. Patients/caregivers are asked 23 questions about their daily activities. The maximum possible score is 78, with a higher score indicating better function

MMSE: assesses six items: orientation, learning, attention, word recall, language use and comprehension, and constructive praxis. A global score from 0 to 30 was computed, with higher scores indicating greater cognitive function (Folstein et al., 1975).

CDR: The CDR is a five-point categorical rating evaluating severity of dementia. The 5-point score is useful for globally staging the level of impairment: 0=No impairment, 0.5, 1, 2, and 3 indicate very mild, mild, moderate and severe dementia respectively, in the following six areas: memory, orientation, judgment, community affairs, home and hobbies, and personal care (Morris J C, 1997).

NPI.: The Neuropsychiatry Inventory assessed the following 10 domains (sub-scales):

delusions, hallucinations, agitation/aggression, depression/dysphoria, anxiety, elation/euphoria, apathy/indifference, disinhibition, irritability/lability and aberrant motor behavior. A global score was computed, with a higher score reflecting a higher level of severity of behavioral problems

Safety was monitored throughout the study, and graded according to the NCI CTCAE v3.0 in all patients receiving at least one dose of masitinib. Safety assessment was based upon the frequency and severity of adverse events (AE), regardless of causality.

Statistical analyses: Efficacy was analyzed in the intent-to-treat (ITT) (all randomized patients, according to group of randomization) and per protocol (PP) (all patients without a major protocol deviation) populations. Datasets examined were Observed Cases (OC), last observed case brought forward (LOCF) and replacement of missing data with treatment failure. Efficacy criteria were analyzed using descriptive statistics, numbers and rates for categorical and discontinuous variables, parametric and non-parametric distribution elements for continuous variables.

Safety was assessed in the safety population (all patients receiving at least one drug administration), with descriptive statistics.

All data analyses and reporting procedures used SAS v9.1 in a Windows XP operating system environment.

Patient disposition: A total of 34 patients were randomized in 12 centers, 26 to masitinib (12 at 3 mg/kg/day and 14 at 6 mg/kg/day) and 8 to placebo. One patient randomized to the 6 mg/kg/day arm was treated in the 3 mg/kg/day arm. A total of 29 patients (22 masitinib, 7 placebo) were considered to be PP. The investigator of Centre #21, which included 8 patients, passed away unexpectedly and no Week 24 measurements were collected for this site.

Nineteen (17 masitinib and 2 placebo) of the 34 (56%) patients treated withdrew before completion of the initial planned 24-week treatment period. Eight from Centre #21 were withdrawn due to the investigator's death. Seven patients (41%) in the masitinib arm withdrew due to related AEs. Two patients were withdrawn due to protocol violations and two patients withdrew consent (one of whom had an AE resulting in an investigator decision to stop treatment).

Fifteen patients (44%) completed the planned initial 24-week treatment period, five (9%) of whom continued in the extension period under blinded treatment (3 placebo, 2 masitinib).

Of the five extension patients, two withdrew due to insufficient therapeutic effect, two receiving placebo were withdrawn at unblinding, and one patient was still on study treatment as of September 2009.

Mean masitinib exposure was longer in the 3 mg/kg/day arm (4.1±2.0 months) than the 6 mg/kg/day group (2.7±1.7 months). Mean duration of treatment for the 8 placebo patients was 5.3±2.2 months.

Patient baseline characteristics are described in Table 1.

TABLE 1
Demographics and clinical characteristics of patients, ITT patients

| | Masitinib | Placebo |
|---|---|---|
| | (N = 26) | (N = 8) |
| Age (years) | | |
| Mean ± Std | 71.8 ± 11.9 | 77.9 ± 10.8 |
| Median | 74.5 | 80.5 |
| Min-Max | 52.0-90.0 | 52.0-86.0 |
| Sex | | |
| Male | 11 (42.3%) | 2 (25.0%) |
| Female | 15 (57.7%) | 6 (75.0%) |
| Age at diagnosis (years) | | |
| Mean ± Std | 69.9 ± 11.8 | 75.6 ± 10.9 |
| Median | 73.0 | 79.0 |
| Min-Max | 51.0-89.0 | 50.0-84.0 |
| Time since diagnosis (years) | | |
| Mean ± Std | 1.7 ± 1.1 | 1.8 ± 0.8 |
| Median | 1.3 | 1.6 |
| Min-Max | 0.5-6.0 | 0.8-3.0 |
| ADCS-ADL | | |
| Missing data | 2 | 1 |
| Mean ± Std | 47.1 ± 11.2 | 45.9 ± 18.0 |
| Median | 46.0 | 42.0 |
| Min-Max | 32.0-71.0 | 24.0-69.0 |
| MMSE | | |
| Slightly severe [10-14] | 3 (11.5%) | 2 (25.0%) |
| Moderate [15-20] | 13 (50.0%) | 3 (37.5%) |
| Mild >= 21 | 10 (38.5%) | 3 (37.5%) |
| Mean ± Std | 19.1 ± 3.9 | 18.0 ± 4.4 |
| Median | 18.5 | 18.0 |
| Min-Max | 13.0-26.0 | 12.0-23.0 |
| CIBIC | | |
| Missing data | 11 | 6 |
| Mild | 6 (40.0%) | 1 (50.0%) |
| Moderate | 8 (53.3%) | 1 (50.0%) |
| Severe | 1 (6.7%) | 0 (0.0%) |
| CDR | | |
| 1 | 21 (80.8%) | 6 (75.0%) |
| 2 | 5 (19.2%) | 2 (25.0%) |
| NPI | | |
| Missing data | 3 | 1 |
| Mean ± Std | 17.4 ± 14.1 | 11.1 ± 7.9 |
| Median | 14.0 | 11.0 |
| Min-Max | 0.0-58.0 | 1.0-25.0 |
| Concomitant AD therapy | | |
| Donepezil | 21 (80.8%) | 5 (62.5%) |
| Rivastigmine | 0 (0.0%) | 1 (12.5%) |
| Galantamine | 5 (19.2%) | 2 (25.0%) |
| Memantine | 4 (15.4%) | 2 (25.0%) |

Results

Efficacy

Results are presented for the ITT population in the OC dataset (with LOCF for Centre #21 at Week 24). Results for the PP population and other datasets showed similar trends.

ADAD-Cog (cognitive function): ADAS-Cog is an 11-item scale that evaluates cognition in Alzheimer's disease patients. A higher rate of responders (decrease ≧4 units) was seen with masitinib compared to placebo from week 8, with 6 of 16 (38%) masitinib patients responding compared to 1 of 5 (20%) placebo patients at week 24. A significantly higher rate of patients with deterioration (increase ≧4 units) was seen under placebo than masitinib at week 12 (p=0.040), and week 24 (p=0.046).

This superiority, albeit non-significant, was also seen in the analysis of ADAS-Cog responders without deterioration in two other parameters (ADCS-ADL and CIBIC-plus); after 12 weeks of treatment, 25% of patients under masitinib met with these response criteria, versus 0% under placebo.

TABLE 2
ADAS-Cog response, Observed Cases, ITT population

| | W 4 | | W 8 | | W 12 | | |
|---|---|---|---|---|---|---|---|
| | AB1010 | placebo | AB1010 | placebo | AB1010 | placebo | pvalue |
| Still ongoing at visit | 26 | 8 | 23 | 8 | 18 | 8 | . |
| Missing | 2 | 2 | 14 | 3 | 1 | 2 | . |
| N | 24 | 6 | 9 | 5 | 17 | 6 | . |
| Response (Decrease >=4) | 3 (12.5%) | 2 (33.3%) | 4 (44.4%) | 1 (20.0%) | 7 (41.2%) | 1 (16.7%) | . |
| No change | 17 (70.8%) | 2 (33.3%) | 4 (44.4%) | 3 (60.0%) | 9 (52.9%) | 2 (33.3%) | . |
| Worsening (Increase >=4) | 4 (16.7%) | 2 (33.3%) | 1 (11.1%) | 1 (20.0%) | 1 (5.9%) | 3 (50.0%) | . |
| Missing | 4 | 3 | 15 | 3 | 2 | 3 | . |
| N | 22 | 5 | 8 | 5 | 16 | 5 | . |
| Response without deterioration | 2 (9.1%) | 0 (0.0%) | 2 (25.0%) | 0 (0.0%) | 4 (25.0%) | 0 (0.0%) | . |
| 3 modalities | | | | | | | 0.044 |
| Response (Decrease >=4) | | | | | | | 0.369 |
| Worsening (Increase >=4) | | | | | | | 0.040 |
| Response without deterioration | | | | | | | 0.532 |

| | W 24 | | W 24 LOCF center 21 | | |
|---|---|---|---|---|---|
| | AB1010 | placebo | AB1010 | placebo | pvalue |
| Still ongoing at visit | 9 | 6 | 16 | 7 | . |
| Missing | 0 | 1 | 0 | 1 | . |
| N | 9 | 5 | 16 | 6 | . |
| Response (Decrease >=4) | 3 (33.3%) | 1 (20.0%) | 6 (37.5%) | 1 (16.7%) | . |
| No change | 5 (55.6%) | 1 (20.0%) | 9 (56.3%) | 2 (33.3%) | . |
| Worsening (Increase >=4) | 1 (11.1%) | 3 (60.0%) | 1 (6.3%) | 3 (50.0%) | . |
| Missing | 1 | 2 | 1 | 2 | . |
| N | 8 | 4 | 15 | 5 | . |
| Response without deterioration | 2 (25.0%) | 1 (25.0%) | 5 (33.3%) | 1 (20.0%) | . |
| 3 modalities | | | | | 0.057 |
| Response (Decrease >=4) | | | | | 0.616 |
| Worsening (Increase >=4) | | | | | 0.046 |
| Response without deterioration | | | | | 1.000 |

A significant difference between the treatment arms was seen in absolute changes in ADAS-Cog score, with a mean increase (i.e. deterioration) of 4.2±6.6 units at Week 12 for placebo patients compared to a mean decrease (i.e. improvement) of 2.6±3.6 units with masitinib (p=0.016); and at week 24, a mean increase of 6.5±8.6 units in placebo patients compared to a mean decrease of 0.7±7.8 units in masitinib-treated patients (p=0.030).

TABLE 3
ADAS-Cog absolute changes, Observed Cases, ITT population

| | W 4 | | W 8 | | W 12 | | | W 24 | | W 24 LOCF center 21 | | |
|---|---|---|---|---|---|---|---|---|---|---|---|---|
| | AB1010 | placebo | AB1010 | placebo | AB1010 | placebo | pvalue | AB1010 | placebo | AB1010 | placebo | pvalue |
| Still | 26 | 8 | 23 | 8 | 18 | 8 | 0.016 | 9 | 6 | 16 | 7 | 0.030 |
| ongoing | | | | | | | | | | | | |
| at visit | | | | | | | | | | | | |
| Missing | 2 | 2 | 14 | 3 | 1 | 2 | . | 0 | 1 | 0 | 1 | . |
| N | 24 | 6 | 9 | 5 | 17 | 6 | . | 9 | 5 | 16 | 6 | . |
| Mean ± | −0.0 ± 6.1 | 2.8 ± 10.0 | −0.8 ± 8.1 | −0.8 ± 6.7 | −2.6 ± 3.6 | 4.2 ± 6.6 | . | −0.7 ± 7.8 | 6.5 ± 8.6 | −1.8 ± 6.1 | 5.8 ± 7.9 | . |
| Std | | | | | | | | | | | | |
| Median | −1.5 | 2.5 | −2.8 | −0.8 | −2.4 | 3.9 | . | −0.3 | 5.3 | −2.5 | 3.9 | . |
| Min; | −7.3; 21.8 | −9.2; 18.6 | −9.1; 17.4 | −10; 8.9 | −7.3; 6.5 | −5.3; 15.3 | . | −13; 16.8 | −5.0; 15.9 | −13; 16.8 | −5.0; 15.9 | . |
| Max | | | | | | | | | | | | |

ASCS-ADL (daily living activities): ADCS-ADL is a scale to assess the impact of a medication-related improvement in everyday functioning and activities (personal hygiene, dressing, eating, shopping, using transportation, handling finances, etc). A higher rate of responders (increase ≧3 units) was seen from Week 8, with 60% of masitinib patients responding at Week 24 versus 17% placebo. This difference was significant in the PP population at Week 12 (p=0.042). A higher rate of patients with deterioration (decrease <0) was reported under placebo at Week 12 (masitinib 31%, placebo 50%) and Week 24 (masitinib 27%, placebo 50%).

TABLE 4
ADCS-ADL response, observed cases, ITT population

| | W 4 | | W 8 | | W 12 | | |
|---|---|---|---|---|---|---|---|
| | AB1010 | placebo | AB1010 | placebo | AB1010 | placebo | pvalue |
| Still ongoing at visit | 26 | 8 | 23 | 8 | 18 | 8 | . |
| Missing | 4 | 3 | 5 | 2 | 2 | 2 | . |
| N | 22 | 5 | 18 | 6 | 16 | 6 | . |
| Response (Increase >=3) | 10 (45.5%) | 2 (40.0%) | 7 (38.9%) | 0 (0.0%) | 8 (50.0%) | 0 (0.0%) | . |
| No change | 5 (22.7%) | 1 (20.0%) | 5 (27.8%) | 4 (66.7%) | 3 (18.8%) | 3 (50.0%) | . |
| Worsening (Decrease <0) | 7 (31.8%) | 2 (40.0%) | 6 (33.3%) | 2 (33.3%) | 5 (31.3%) | 3 (50.0%) | . |
| 3 modalities | | | | | | | 0.100 |
| Response (Increase >=3) | | | | | | | 0.051 |
| Worsening (Decrease <0) | | | | | | | 0.624 |

| | W 24 | | W 24 LOCF center 21 | | |
|---|---|---|---|---|---|
| | AB1010 | placebo | AB1010 | placebo | pvalue |
| Still ongoing at visit | 9 | 6 | 16 | 7 | . |
| Missing | 1 | 1 | 1 | 1 | . |
| N | 8 | 5 | 15 | 6 | . |
| Response (Increase >=3) | 5 (62.5%) | 1 (20.0%) | 9 (60.0%) | 1 (16.7%) | . |
| No change | 0 (0.0%) | 1 (20.0%) | 2 (13.3%) | 2 (33.3%) | . |
| Worsening (Decrease <0) | 3 (37.5%) | 3 (60.0%) | 4 (26.7%) | 3 (50.0%) | . |
| 3 modalities | | | | | 0.129 |
| Response (Increase >=3) | | | | | 0.149 |
| Worsening (Decrease <0) | | | | | 0.354 |

A significant difference in mean absolute changes relative to baseline (increase=improvement) was seen at Week 12 (masitinib 6.9±10.9, placebo −4.2±6.9; p=0.035), and Week 24 (masitinib 5.5±15.8; placebo −1.8±7.0, not significant).

TABLE 5
ADCS-ADL absolute change relative to baseline, Observed Cases, ITT population

| | W 4 | | W 8 | | W 12 | | | W 24 | | W 24 LOCF center 21 | | |
|---|---|---|---|---|---|---|---|---|---|---|---|---|
| | AB1010 | placebo | AB1010 | placebo | AB1010 | placebo | pvalue | AB1010 | placebo | AB1010 | placebo | pvalue |
| Still | 26 | 8 | 23 | 8 | 18 | 8 | 0.035 | 9 | 6 | 16 | 7 | 0.128 |
| ongoing | | | | | | | | | | | | |
| at visit | | | | | | | | | | | | |
| Missing | 4 | 3 | 5 | 2 | 2 | 2 | . | 1 | 1 | 1 | 1 | . |
| N | 22 | 5 | 18 | 6 | 16 | 6 | . | 8 | 5 | 15 | 6 | . |
| Mean ± | 1.8 ± 6.1 | −0.6 ± 3.9 | 0.3 ± 7.0 | −3.0 ± 7.5 | 6.9 ± 10.9 | −4.2 ± 6.9 | . | −1.4 ± 15.3 | −2.6 ± 7.5 | 5.5 ± 15.8 | −1.8 ± 7.0 | . |
| Std | | | | | | | | | | | | |
| Median | 1.5 | 0.0 | 1.0 | 0.5 | 3.0 | −2.0 | . | 4.0 | −1.0 | 4.0 | 0.0 | . |
| Min; | −9.0; 14.0 | −6.0; 3.0 | −16; 11.0 | −17; 2.0 | −6.0; 31.0 | −17; 2.0 | . | −37; 10.0 | −15; 5.0 | −37; 31.0 | −15; 5.0 | . |
| Max | | | | | | | | | | | | |

CIBIC-Plus (global clinical assessment): CIBIC-plus is a global assessment scale that allows assessment of global clinical status of the patient relative to baseline. At Week 12 more placebo patients showed worsening than masitinib-treated patients (50% versus 12%).

TABLE 6
CIBIC-plus response, Observed Cases, ITT population

| | W 4 | | W 8 | | W 12 | | |
|---|---|---|---|---|---|---|---|
| | AB1010 | placebo | AB1010 | placebo | AB1010 | placebo | pvalue |
| Still ongoing at visit | 26 | 8 | 23 | 8 | 18 | 8 | . |
| Missing | 2 | 1 | 4 | 2 | 1 | 2 | . |
| N | 24 | 7 | 19 | 6 | 17 | 6 | . |
| Response (CIBIC in [1-3]) | 4 (16.7%) | 1 (14.3%) | 2 (10.5%) | 0 (0.0%) | 1 (5.9%) | 1 (16.7%) | . |
| No change (CIBIC = 4) | 16 (66.7%) | 4 (57.1%) | 16 (84.2%) | 5 (83.3%) | 14 (82.4%) | 2 (33.3%) | . |
| Worsening (CIBIC in [5-7]) | 4 (16.7%) | 2 (28.6%) | 1 (5.3%) | 1 (16.7%) | 2 (11.8%) | 3 (50.0%) | . |
| 3 modalities | | | | | | | 0.292 |
| Response (CIBIC in [1-3]) | | | | | | | 0.462 |
| Worsening (CIBIC in [5-7]) | | | | | | | 0.089 |

| | W 24 | | W 24 LOCF center 21 | | |
|---|---|---|---|---|---|
| | AB1010 | placebo | AB1010 | placebo | pvalue |
| Still ongoing at visit | 9 | 6 | 16 | 7 | . |
| Missing | 0 | 1 | 0 | 1 | . |
| N | 9 | 5 | 16 | 6 | . |
| Response (CIBIC in [1-3]) | 2 (22.2%) | 0 (0.0%) | 2 (12.5%) | 0 (0.0%) | . |
| No change (CIBIC = 4) | 5 (55.6%) | 4 (80.0%) | 12 (75.0%) | 5 (83.3%) | . |
| Worsening (CIBIC in [5-7]) | 2 (22.2%) | 1 (20.0%) | 2 (12.5%) | 1 (16.7%) | . |
| 3 modalities | | | | | 0.474 |
| Response (CIBIC in [1-3]) | | | | | 1.000 |
| Worsening (CIBIC in [5-7]) | | | | | 1.000 |

MMSE (global clinical assessment): The MMSE is a 30-point composite clinical test that detects cognitive impairments.

Scores in masitinib-treated patients remained stable over time, while placebo-treated patients showed a deterioration in cognitive function (Week 12 p=0.047, Week 24 p=0.031).

TABLE 7
MMSE score, absolute change relative to baseline, Observed Cases, ITT population

| | W 4 | | W 8 | | W 12 | | | W 24 | | W 24 LOCF center 21 | | |
|---|---|---|---|---|---|---|---|---|---|---|---|---|
| | AB1010 | placebo | AB1010 | placebo | AB1010 | placebo | pvalue | AB1010 | placebo | AB1010 | placebo | pvalue |
| Ongoing | 26 | 8 | 23 | 8 | 18 | 8 | 0.047 | 9 | 6 | 16 | 7 | 0.031 |
| Missing | 2 | 2 | 14 | 2 | 1 | 1 | . | 0 | 0 | 0 | 0 | . |
| N | 24 | 6 | 9 | 6 | 17 | 7 | . | 9 | 6 | 16 | 7 | . |
| Mean ± | 0.2 ± 2.0 | −1.5 ± 2.9 | −0.6 ± 2.8 | −1.8 ± 2.6 | 0.1 ± 2.5 | −2.1 ± 2.5 | . | −0.4 ± 5.6 | −3.8 ± 3.2 | −0.1 ± 4.3 | −3.3 ± 3.3 | . |
| Std | | | | | | | | | | | | |
| Median | −0.5 | −0.5 | 0.0 | −1.5 | 0.0 | −1.0 | . | 1.0 | −3.5 | 0.0 | −3.0 | . |
| Min; | −2.0; 5.0 | −6.0; 2.0 | −4.0; 5.0 | −6.0; 1.0 | −5.0; 4.0 | −7.0; 0.0 | . | −14; 4.0 | −8.0; 0.0 | −14; 4.0 | −8.0; 0.0 | . |
| Max | | | | | | | | | | | | |

CDR (global clinical assessment): The CDR is a 5-point scale used to characterize six domains of cognitive and functional performance applicable to AD and related dementias: Memory, Orientation, Judgment & Problem Solving, Community Affairs, Home & Hobbies, and Personal Care.

The overall CDR score, calculated through the use of an algorithm, is useful for characterizing and tracking a patient's level of impairment/dementia: 0=Normal, 0.5=Very Mild Dementia, 1=Mild Dementia, 2=Moderate Dementia, 3=Severe Dementia.

A significant difference in absolute CDR score between the two treatment arms was reported (p=0.018, Week 24). Frequency of patients with lower scores (i.e. milder impairment), was higher in masitinib-treated patients (Score 0.5-1 at Week 24: masitinib 94%, placebo 43%).

TABLE 8
CDR - ITT population (n = 34) - Observed Cases

| | W 4 | | W 8 | | W 12 | | |
|---|---|---|---|---|---|---|---|
| | AB1010 | placebo | AB1010 | placebo | AB1010 | placebo | pvalue |
| Ongoing | 26 | 8 | 23 | 8 | 18 | 8 | . |
| Missing | 16 | 4 | 15 | 4 | 1 | 1 | . |
| N | 10 | 4 | 8 | 4 | 17 | 7 | . |
| 0 | 0 (0.0%) | 0 (0.0%) | 0 (0.0%) | 0 (0.0%) | 0 (0.0%) | 0 (0.0%) | . |
| 0.5 | 0 (0.0%) | 0 (0.0%) | 0 (0.0%) | 0 (0.0%) | 1 (5.9%) | 1 (14.3%) | . |
| 1 | 7 (70.0%) | 2 (50.0%) | 6 (75.0%) | 2 (50.0%) | 15 (88.2%) | 3 (42.9%) | . |
| 2 | 3 (30.0%) | 2 (50.0%) | 2 (25.0%) | 2 (50.0%) | 1 (5.9%) | 3 (42.9%) | . |
| 3 | 0 (0.0%) | 0 (0.0%) | 0 (0.0%) | 0 (0.0%) | 0 (0.0%) | 0 (0.0%) | . |
| p-value | | | | | | | 0.085 |

| | W 24 | | W 24 LOCF center 21 | | |
|---|---|---|---|---|---|
| | AB1010 | placebo | AB1010 | placebo | pvalue |
| Ongoing | 9 | 6 | 16 | 7 | . |
| Missing | 0 | 0 | 0 | 0 | . |
| N | 9 | 6 | 16 | 7 | . |
| 0 | 0 (0.0%) | 0 (0.0%) | 0 (0.0%) | 0 (0.0%) | . |
| 0.5 | 2 (22.2%) | 1 (16.7%) | 2 (12.5%) | 1 (14.3%) | . |
| 1 | 6 (66.7%) | 1 (16.7%) | 13 (81.3%) | 2 (28.6%) | . |
| 2 | 1 (11.1%) | 3 (50.0%) | 1 (6.3%) | 3 (42.9%) | . |
| 3 | 0 (0.0%) | 1 (16.7%) | 0 (0.0%) | 1 (14.3%) | . |
| p-value | | | | | 0.018 |

At Week 24, a non-significant superiority trend in favor of masitinib was observed in the CDR score. After 24 weeks of treatment, masitinib was associated with a worsening of the CDR score in 6.3% of patients, versus 28.6% or the placebo.

TABLE 9
CDR - Response Rate - ITT population (n = 34) - Observed Cases

| | W 4 | | W 8 | | W 12 | | |
|---|---|---|---|---|---|---|---|
| | AB1010 | placebo | AB1010 | placebo | AB1010 | placebo | pvalue |
| Ongoing | 26 | 8 | 23 | 8 | 18 | 8 | . |
| Missing | 16 | 4 | 15 | 4 | 1 | 1 | . |
| N | 10 | 4 | 8 | 4 | 17 | 7 | . |
| Response (Decrease >0) | 0 (0.0%) | 0 (0.0%) | 0 (0.0%) | 0 (0.0%) | 2 (11.8%) | 1 (14.3%) | . |
| No change | 9 (90.0%) | 4 (100%) | 7 (87.5%) | 4 (100%) | 14 (82.4%) | 5 (71.4%) | . |
| Worsening (Increase >0) | 1 (10.0%) | 0 (0.0%) | 1 (12.5%) | 0 (0.0%) | 1 (5.9%) | 1 (14.3%) | . |
| 3 modalities | | | | | | | 0.778 |
| Response (Decrease >0) | | | | | | | 1.000 |
| Worsening (Increase >0) | | | | | | | 0.507 |

| | W 24 | | W 24 LOCF center 21 | | |
|---|---|---|---|---|---|
| | AB1010 | placebo | AB1010 | placebo | pvalue |
| Ongoing | 9 | 6 | 16 | 7 | . |
| Missing | 0 | 0 | 0 | 0 | . |
| N | 9 | 6 | 16 | 7 | . |
| Response (Decrease >0) | 2 (22.2%) | 1 (16.7%) | 3 (18.8%) | 1 (14.3%) | . |
| No change | 6 (66.7%) | 3 (50.0%) | 12 (75.0%) | 4 (57.1%) | . |
| Worsening (Increase >0) | 1 (11.1%) | 2 (33.3%) | 1 (6.3%) | 2 (28.6%) | . |
| 3 modalities | | | | | 0.293 |
| Response (Decrease >0) | | | | | 1.000 |
| Worsening (Increase >0) | | | | | 0.209 |

Safety:
Seventeen of 26 masitinib-treated patients (65%) experienced at least one AE, considered related for 15 of these patients (58%). Three of the eight placebo-treated patients (38%) experienced at least one AE, all of which were considered related.
No deaths were reported. Five of the 26 masitinib-treated patients (19%) experienced at least one related serious adverse event.
Nine masitinib-treated patients (35%) had related AEs leading to treatment discontinuation, and four patients treated with masitinib had dose reductions due to AEs, all of which were considered related.
The majority of AEs were mild to moderate in intensity, with four masitinib (15%) patients and one placebo (13%) patient experiencing a severe AE. All four severe AEs in masitinib-treated patients were considered related.
The most frequent (>10% of patients) AEs with their causalities are listed in Table 5.
Seven masitinib-treated patients (27%) experienced edema, 15% rash, 27% nausea/vomiting and 23% had diarrhea. For all but two patients (with nausea/vomiting), these events were considered related. One patient experienced severe transaminase elevation.
The frequency of overall and individual AEs was similar in the two masitinib treatment arms (3 and 6 mg/kg/day), suggesting there was no dose effect in terms of toxicity.

TABLE 10
Adverse events occurring in ≧10% of patients

| | Masitinib (N = 26) | | | | | | Placebo (N = 8) | | |
|---|---|---|---|---|---|---|---|---|---|
| | All | | Moderate | | Severe | | All | Moderate | Severe |
| At least one AE | 17 | (65.4%) | 11 | (42.3%) | 4 | (15.4%) | 3 (37.5%) | 2 (25.0%) | 1 (12.5%) |
| Hematological | | | | | | | | | |
| Anemia | 13 | (50.0%) | 2 | (7.7%) | | | 1 (12.5%) | | |
| Leukopenia | 7 | (26.9%) | 1 | (3.8%) | | | | | |
| Neutropenia | 11 | (42.3%) | | | | | 2 (25.0%) | | |
| Thrombocytopenia | 4 | (15.4%) | | | | | | | |
| Non hematological | | | | | | | | | |
| Diarrhea | 6 | (23.1%) | 3 | (11.5%) | | | | | |
| Peripheral edema | 5 | (19.2%) | 3 | (11.5%) | | | | | |
| Eyelid edema | 4 | (15.4%) | 2 | (7.7%) | | | | | |
| Anorexia | 4 | (15.4%) | 2 | (7.7%) | 2 | (7.7%) | | | |
| Nausea | 4 | (15.4%) | 1 | (3.8%) | 1 | (3.8%) | | | |
| Vomiting | 3 | (11.5%) | 2 | (7.7%) | | | | | |
| Asthenia | 3 | (11.5%) | 1 | (3.8%) | 1 | (3.8%) | | | |
| Weight Decreased | 2 | (7.7%) | 2 | (7.7%) | | | 1 (12.5%) | 1 (12.5%) | |
| Hyperuricemia | | | | | | | 1 (12.5%) | | |
| Balance Disorder | | | | | | | 1 (12.5%) | | 1 (12.5%) |
| Depression | 2 | (7.7%) | | | | | 1 (12.5%) | 1 (12.5%) | |
| Proteinuria | | | | | | | 1 (12.5%) | | |
| Hypertension | | | | | | | 1 (12.5%) | 1 (12.5%) | |

Conclusion

This exploratory double-blind, multicenter, randomized, phase 2 study, was conducted in a limited number of patients suffering from mild to moderate Alzheimer's disease who were treated with masitinib at a starting dose of 3 or 6 mg/kg/day or placebo, in association with the current standard of care in Alzheimer's disease, i.e. a acetylcholinesterase inhibitor and/or the NDMA inhibitor memantine.

In this study, masitinib shows efficacy in the treatment of patients suffering from mild to moderate Alzheimer disease, particularly in the improvement of cognitive functions (ADAS-Cog score over placebo), and the functional domain (statistically significant improved ADCS-ADL score over placebo). A statistically significant improvement was also obtained with masitinib on the MMSE and CDR scores.

Analysis of the safety data shows that the tolerance of masitinib in phase 2 study in this elderly population suffering from Alzheimer's disease is similar to that observed in other non-oncology indications. In particular, the profile of the most frequent AEs reported (edema, nausea/vomiting, rash, diarrhea) is similar to those observed in other phase 2 non-oncology studies and can be explained by the mechanism of action of masitinib. Anorexia is probably the consequence of digestive disorders. No patients died and nine of the masitinib-treated patients (35%) withdrew from their treatment due to masitinib-related AEs. Of note, the majority of AEs were mild to moderate in intensity, and the incidence and frequency was similar in the two treatment arms (3.0 and 6.0 mg/kg/day).

In conclusion, these results and the risk/benefit balance in this indication are promising for treatment of human patients with mild to moderate Alzheimer's disease with masitinib at 3.0 to 6.0 mg/kg/day in association with a acetylcholinesterase inhibitor and/or memantine.

### REFERENCES

Aricept Label Information. Eisai Inc., Teaneck, N.J., US.

Brookmeyer R, Johnson E, Ziegler-Graham K, and Arrighi H M. “Forecasting the Global Burden of Alzheimer's Disease” Alzheimer's and Dementia 3.3 (2007): 186-191.

Dubreuil P, Letard S, Ciufolini M, Gros L, Humbert M, Castéran N, Borge L, Hajem B, Lermet A, Sippl W, Voisset E, Arock M, Auclair C, Leventhal P S, Mansfield C D, Moussy A, Hermine O (2009) Masitinib (AB1010), a potent and selective tyrosine kinase inhibitor targeting KIT. PLoSONE 4(9): e7258. doi:10.1371/journal.pone.0007258.

Exelon Label Information (2006). Novartis Pharmaceuticals Corporation, East Hanover, N.J., US.

Folstein M F, Folstein S E, McHugh P R (1975). Mini-mental state. A practical method for grading the cognitive state of patients for the clinician. Journal of psychiatric research 12 (3): 189-98. doi:10.1016/0022-3956(75)90026-6. PMID 1202204.

Lee G, Thangavel R, Sharma V M, Litersky J M, Bhaskar K, Fang S M, Do L H, Andreadis A, Van Hoesen G, Ksiezak-Reding H. Phosphorylation of Tau by Fyn: Implications for Alzheimer's Disease; The Journal of Neuroscience, Mar. 3, 2004, 24(9):2304-2312.

Morris J C (1997) Clinical Dementia Rating: A Reliable and Valid Diagnostic and Staging Measure for Dementia of the Alzheimer Type. International Psychogeriatrics, Volume 9, Supplement 51, December 1997, pp 173-176 doi: 10.1017/S1041610297004870.

Namenda Label Information (2007). Forest Pharmaceuticals, Inc. St. Louis, Mo., US.

St George-Hyslop PH Sci Am. Piecing together Alzheimer's. December 2000; 283(6):76-83.

Veld B A, Ruitemberg A, Hofman A, Launer L J, van Duijn C M, Stijnen T et al. Nonsteroidal antiinflammatory drugs and the risk of Alzheimer's disease. N Engl J Med 2001; 345:1515-21.

1. (canceled)

2. A method of treatment of dementia of Alzheimer's type according to the classification of the Diagnostic and Statistical Manual—Revision 4 (DSM IV criteria) or according to the probable Alzheimer's disease criteria of the National Institute of Neurological and Communicative Disorders and Stroke and the Alzheimer's Disease and Related Disorders Association (NINCDS-ADRDA), comprising administering masitinib or a pharmaceutically acceptable salt thereof and at least one of NMDA (N-methyl-D-aspartic acid) receptor antagonists and acetylcholinesterase inhibitors to human patients, wherein masitinib is to be administered daily at a starting dose of 3.0 to 6.0±1.5 mg/kg/day, optionally combined with at least one of NMDA (N-methyl-D-aspartic acid) receptor antagonists and acetylcholinesterase inhibitors, and wherein said patients are between 9 to 26 to the mini-mental state examination (MMSE).

3. The use or the method according to claim 2, wherein said patients are between 10 to 26 to the mini-mental state examination (MMSE).

4. The use or the method according to claim 2, wherein said patients are between 0.5 and 2 in the CDR scale.

5. The use or the method according to claim 2, wherein masitinib is masitinib mesilate.

6. The use or the method according to claim 2, wherein masitinib is to be administered at a starting daily dose of 3.0 to 6.0 mg/kg/day.

7. The use or the method according to claim 2, wherein masitinib is dose escalated by increments of 1.5 mg/kg/day to reach a maximum of 7.5 mg/kg/day.

8. The use or the method according to claim 2, wherein patients are those afflicted with dementia of mild to moderately severe Alzheimer's type, specifically with an MMSE score of 10-26 and with a CDR score between 0.5 to 2.

9. The use or the method according to claim 2, wherein masitinib, or salts thereof, are administered orally.

10. The use or the method according to claim 2 wherein masitinib is administered twice a day.

11. The use or the method according to claim 2 comprising a long-term administration of an effective amount of masitinib over more than 6 months, preferably more than 12 months.

12. The use or the method according to claim 2 wherein the NMDA receptor antagonist is memantine.

13. The use or method according to claim 12, wherein memantine is to be administered between 5 to 20 mg/day.

14. The use or the method according to claim 2 wherein the acetylcholinesterase inhibitor is selected from donepezil, rivastigmine and galantamine.

15. The use or method according to claim 13, wherein donepezil is to be administered between 5 to 10 mg/day, rivastigmine is to be administered between 3 to 12 mg/day, or galantamine is to be administered between 8 to 24 mg/day.

16. The use or the method according to claim 2 wherein masitinib and at least one of NMDA receptor antagonist and acetylcholinesterase inhibitor are to be administered separately, simultaneously or sequentially in time.
